(19) United States
(12) Patent Application Publication
(10) Pub. No.: US 2003/0194420 A1
(43) Pub. Date: Oct. 16, 2003

(54) Process for loading a drug delivery device

(76) Inventors: Richard Holl, Ft. Collins, CO (US); Kasey Kravig, Loveland, CO (US); Scott Jeffers, Fort Collins, CO (US); David W. Osborne, Fort Collins, CO (US); A. Gerald Beaudoin, Perry, OH (US); Amy Poshusta, Broomfield, CO (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/121,430

(22) Filed: Apr. 11, 2002

(51) Int. Cl. A61K 9/00

(52) U.S. Cl. 424/426

(57) ABSTRACT

The invention provides methods for incorporating one or more compositions, each containing at least one active ingredient, into a preformed water-soluble pharmaceutical carrier device. Sustained delivery devices and methods of using such devices are also provided.

### FIELD OF THE INVENTION

The present invention relates generally to methods of incorporating a composition onto a bioerodible, water-soluble pharmaceutical carrier device to efficiently manufacture a device that can optimally deliver the composition either systemically or locally.

### BACKGROUND OF THE INVENTION

A number of mucoadhesive devices are available for the delivery of active agents locally or systemically through a mucus membrane or within a mucosally lined body cavity. Many of these devices are in the form of a film or patch that conveniently fit within a cavity, commonly the mouth, and adhere to a mucus membrane. They are commonly designed to be pressure sensitive, and they adhere immediately upon application to a membrane.

The BEMA™ (Bioerodible Muco-Adhesive Film) Drug Delivery System is a bioerodible film for fast-acting local or systemic delivery of active agents. The BEMA™ technology provides a mucoadhesive and bioerodible disc for application to a mucosal surface and is used for transmucosal delivery of active agents over variable lengths of time, for example, delivery occurring for minutes or hours. The BEMA technology is disclosed in Tapolsky, et al. (U.S. Pat. No. 5,800,832) and Tapolsky, et al. (U.S. Pat. No. 6,159,498) the entire contents of which are hereby incorporated by reference.

The method of manufacturing a product using the BEMA™ Delivery System involves adding an active agent into the bioadhesive mixture used to produce the bioadhesive layer, into the non-bioadhesive backing mixture used to produce the backing layer, or into both the bioadhesive mixture and non-bioadhesive backing mixture. In any of these techniques the mixtures containing an active agent are coated sequentially to form a layered film. This process has been termed the “preloading” manufacturing technique.

Although the preloading technique for manufacturing the BEMA™ Delivery System is suitable for a number of active agents, it can lead to practical limitations and difficulties when certain active agents are used in the manufacture of BEMA™ products. A significant limitation can be a chemical or physical interaction between an incorporated active agent and one or more of the ingredients of the bioadhesive or non-bioadhesive mixtures. An incompatibility between an active agent and a film ingredient can result in the failure of one or both mixtures to form a uniform and homogeneous film during the manufacturing process. For example, an ionic interaction between an active agent and an ingredient in the mixture prepared for forming either the bioadhesive layer or the backing layer can result in precipitation of the active agent. In such a case the active agent cannot be uniformly distributed within the material, and it is not possible to form the film layers of the BEMA™ Delivery System. Such incompatibilities can prevent entire chemical classes of active agents from being incorporated within and delivered with a mucoadhesive device.

Other practical difficulties of the preloading manufacturing technique include diminished cost effectiveness as a result of the loss of active agents in the scrap generated after cutting the BEMA™ discs from the bilayer film. Further, when the active agent is a controlled substance, substantial losses of expensive pharmaceutical compounds can occur. The high expense in the latter case is compounded since the manufacturer is required to contain and account for controlled substance pharmaceuticals during the necessary cutting and trimming steps required for BEMA™ disc formation.

A method of manufacture that circumvents the need to incorporate an active agent into the layered material would eliminate the limitations associated with producing such mucoadhesive devices.

Some disclosures describe single or multi-layered mucoadhesive films for the delivery of active agents, but none have the structure contemplated for the devices of the invention. Nor do they address or overcome the potential limitations outlined above. See Chien, et al. (U.S. Pat. No. 5,578,315); Biegajski, et al. (U.S. Pat. No. 5,700,478); Rault, et al. (U.S. Pat. No. 5,900,247); Zerbe, et al. (U.S. Pat. No. 5,948,430) Acharya et al. (U.S. Pat. No. 6,210,699); Ebert, et al. (U.S. Pat. No. 5,626,866).

In general, transmucosal delivery devices are manufactured with an active agent incorporated with the polymer mixture prior to the formation of a film.

The methodology of loading active agents onto transdermal delivery devices, in contrast to transmucosal devices, is more established but the structural differences between transdermal devices and transmucosal devices do not support the easy transfer of the established methodologies. A common technique involves the depositing or printing of an active agent in liquid form onto an adsorbent fabric layer of the transdermal device. Miranda, et al. (U.S. Pat. No. 4,915,950) and Hoffmann (U.S. Pat. No. 6,139,868) disclosed variations of this technique. In general such an absorbent fabric is used or an added processing step is needed to drive the active substance into the device. Haralambopoulos (U.S. Pat. No. 5,965,154)

A method for the manufacture of a mucoadhesive, transmucosal delivery device that eliminates the need to incorporate the active agent into a pre-film polymer mixture would solve problems associated with the preloading technique. A technique suitable for depositing an active material onto a transmucosal film and incorporating the deposited material into a finished film product would permit the production of devices for the delivery of additional classes of active and pharmaceutical agents.

### SUMMARY OF THE INVENTION

The invention provides a method of incorporating an effective amount of a composition containing at least one active ingredient to a bioerodible, water-soluble pharmaceutical carrier device. The method for incorporating a composition onto a pre-formed bioerodible, water-soluble carrier device generally includes: depositing the composition onto a surface of the preformed bioerodible, water-soluble carrier device to form a loaded bioerodible, water-soluble, carrier device. Several deposits of the same or of a number of different compositions can be incorporated onto the preformed bioerodible, water-soluble carrier device. The delivery of active ingredients within the composition(s) can be facilitated and modulated by using multiple deposits.

The invention provides a method for incorporating a composition into a preformed bioerodible, water-soluble carrier device. The method includes depositing the composition onto at least one surface of the preformed bioerodible, water-soluble carrier device to form a loaded bioerodible, water-soluble carrier device; wherein the bioerodible, water-soluble carrier device comprises at least a bioadhesive layer and a non-bioadhesive backing layer. In general, the composition is preferably deposited onto a surface of the bioadhesive layer.

Hence, in another embodiment, the method involves depositing the composition onto a surface of the bioadhesive layer of the preformed bioerodible, water-soluble carrier device to form a loaded bioerodible, water-soluble carrier device.

In another embodiment, the method involves incorporating at least one composition comprising at least one active ingredient into a preformed bioerodible, water-soluble carrier device, where the method involves combining the at least one active ingredient with a fluid carrier to form a composition wherein the fluid carrier is selected from materials suitable for administration to a mucosal surface, depositing one or more portions of said composition onto the preformed bioerodible, water-soluble carrier device, and allowing said composition to form at least one deposit layer on the preformed bioerodible, water-soluble carrier device to form a loaded bioerodible, water-soluble carrier device.

In another embodiment, the method involves combining at least one active agent with a film-forming material to form a solid film composition; and laminating said solid film composition onto a surface of a layer of the preformed bioerodible, water-soluble carrier device to form a loaded the bioerodible, water-soluble carrier device.

The bioerodible, water-soluble, carrier device of the invention comprises a non-bioadhesive backing layer, a bioadhesive layer and a composition comprising an active ingredient, wherein the composition is deposited onto a surface of the bioerodible, water-soluble, carrier device. A surface of either the non-bioadhesive backing layer or the bioadhesive layer can be used for deposit of the composition onto the bioerodible, water-soluble, carrier device. The bioadhesive layer of the device can adhere to a mucosal surface of a mammal. In some embodiments, the composition does not cover the entire surface of the layer. For example, the composition can be deposited near the center of the bioadhesive layer allowing the periphery of the bioadhesive layer to optimally adhere to a mucosal surface of a mammal. After application to the mucosal surface of a mammal, the device can provide sustained delivery of the composition.

The bioadhesive layer is generally water-soluble and can be made from a film forming water-soluble polymer and a bioadhesive polymer. The non-bioadhesive backing layer is also water-soluble and can include a pharmaceutically acceptable, water-soluble, film-forming polymer. The non-bioadhesive backing layer will dissolve first after application of the device to a mucosal surface of the mammal.

Compositions incorporated into the bioerodible, water-soluble carrier device of the invention can be liquid, solid, suspension, molten or powder compositions when deposited onto either surface of the device. Such compositions can include any pharmaceutical, drug, or active ingredient selected by one of skill in the art. The composition(s) can be deposited onto either surface or layer more than once, for example, in some embodiments the composition is deposited onto either surface or layer between about 1 to about 10 times.

The composition loaded into the bioerodible, water-soluble, carrier device can include any active ingredient, pharmaceutical, or excipient selected by one of skill in the art. Examples are provided throughout the application. The composition generally comprises between about 0.001 percent and about 50 percent by weight of the loaded bioerodible, water-soluble, carrier device. In other embodiments, the composition comprises between about 0.005 percent and about 35 percent by weight of the loaded bioerodible, water-soluble, carrier device.

A fluid carrier can be employed to suspend or dissolve an active ingredient of the composition. Such a fluid carrier can be a liquid carrier. Preferred liquid carriers are pharmaceutically acceptable solvents suitable for oral administration. Liquid carriers are also preferably volatile liquids, for example, those with low boiling points. Examples of fluid carriers include acetic acid, acetone, anisole, 1-butanol, 2-butanol, butyl acetate, tertbutylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Other components can be included within the composition, such as additional active ingredients, carriers, excipients and the like. For example, the composition can include a viscosity-building agent or more than one active ingredient.

According to the invention, multiple deposits of the same or different composition(s) on the device can lead to more desirable sustained release properties. Hence, composition(s) can also be deposited more than once onto one or more selected surfaces of the device. For example, the depositing or laminating step can be performed 1 to 10 times. Moreover, several different compositions can be deposited or laminated sequentially, alternatively or repeatedly onto one or the other of the surfaces of the device. For example, 2 to 10 different compositions can be deposited or laminated onto the device in any order or combination selected by one of skill in the art.

The invention is also directed to bioerodible, water-soluble, carrier devices made by the method provided herein.

In another embodiment, the invention provides a method for sustained delivery of a pharmaceutical composition to a mammal that comprises applying a bioerodible, water-soluble, carrier device to a mucosal surface of the mammal, wherein the bioerodible, water-soluble, carrier device comprises a non-bioadhesive backing layer, a bioadhesive layer and a composition comprising an active ingredient, and wherein the composition is deposited onto either the non-bioadhesive backing layer or the bioadhesive layer after formation of the bioerodible, water-soluble, carrier device. When the composition is a liquid, the depositing step can further comprise drying the composition onto the bioadhesive layer. The depositing step can be performed more than once to form a loaded bioerodible, water-soluble, carrier device. In fact, the composition can be deposited onto the bioadhesive layer multiple times. For example, in some embodiments the composition is deposited onto the bioadhesive layer between about 1 to about 10 times.

### DESCRIPTION OF THE FIGURES

FIG. 1 provides the plasma concentrations of ondansetron after application of preloaded and post-loaded BEMA™-Ondansetron discs.

FIG. 2 provides the plasma hydrocodone concentrations (mean±standard error) after application to the mucosal surfaces of dogs of preloaded BEMA™-Hydrocodone discs (7.2 mg/disc hydrocodone bitartrate, open circles) and post-loaded BEMA™-Hydrocodone discs (3 mg/disc formed by 3 discrete deposits of hydrocodone free base, closed circles).

FIG. 3 provides plasma hydrocodone concentrations (mean±standard error) after application of post-loaded BEMA™-Hydrocodone discs with 1 discrete post-load deposit (filled triangles), 3 discrete post-load deposits (filled circles), or 5 discrete post-load deposits (open circles).

FIG. 4 illustrates the post-loading method of the invention. As illustrated, the bioerodible, water-soluble carrier device has a non-bioadhesive backing layer 1 and a bioadhesive layer 2. The method involves depositing a composition onto a surface of the bioerodible, water-soluble, carrier device, for example, the composition can be deposited onto the surface of the bioadhesive layer 2 of the pre-formed bioerodible, water-soluble carrier device to form a loaded bioerodible, water-soluble, carrier device having a composition deposit 3 on the bioadhesive layer.

### DETAILED DESCRIPTION OF THE INVENTION

The invention provides a method for loading a composition into a pre-assembled water-soluble, bioerodible carrier device that can adhere to mucosal surfaces. The method generally involves applying the desired amount of the composition onto one or two surfaces of the water-soluble carrier device. For pharmaceutical compositions, a therapeutically effective amount of an active ingredient(s) or pharmaceutical(s) can be deposited onto one or more surfaces of the water-soluble carrier device. The composition can be applied to the chosen surface(s) in the form of a liquid or solid. After applying the composition to form a loaded device, the bioadhesive layer of the bioerodible carrier device can be placed in contact with a mucosal surface of a mammal for delivery of the active ingredient(s) or pharmaceutical(s) within the composition.

The composition to be incorporated into the water-soluble, bioerodible pharmaceutical device can be applied as a liquid in the form of a solution, suspension or melted composition, or as a solid in the form of a powder, film or tablet. The composition is prepared with the active ingredient(s) or pharmaceutical(s) and any pharmaceutically acceptable excipients selected by one of skill in the art. Any convenient excipient can be included into the solution. Examples include, but are not limited to, viscosity-building agents (both polymeric and nonpolymeric), hydrophilicity agents (both polymeric and nonpolymeric), coloring agents and other excipients described herein. Examples of a viscosity-building agents include hydroxypropylcellulose and hydroxyethylcellulose. An example of a hydrophilicity agent is polyethylene glycol.

The solvent used for the solution or suspension can vary and depends upon the active ingredient(s) or pharmaceutical(s) employed as well as the other components of the composition. In general, one of skill in the art can select a good solvent for the composition to be incorporated into the water-soluble, bioerodible pharmaceutical device. Preferred solvents include organic-based solvents that have a high vapor pressure or a low normal boiling point and that have regulatory acceptance as a pharmaceutical solvent suitable for oral administration. Examples of solvents useful for preparing and dispensing a solution or suspension of active ingredient(s) or pharmaceutical(s) include, for example, acetic acid, acetone, anisole, 1-butanol, 2-butanol, butyl acetate, tert-butylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. Preferred solvents that may be used include methanol, ethanol or isopropanol.

In general, the amount of water in the solvent should be kept to a minimum to prevent the composition from running off of the device during application and to prevent dissolution of the water-soluble components of the device.

Molten compositions can also be incorporated onto the device. In general, such molten compositions are used when the active ingredient(s) or pharmaceutical(s) are stable at the temperatures needed to melt the composition. Molten compositions can be easily and accurately dispensed onto the device. Once incorporated onto the device, the molten composition can quickly solidify without significant diffusion into, or off of, the device.

If the composition is to be deposited in a solid form, a solid form is prepared from the composition that contains the active ingredient(s) or pharmaceutical(s) and acceptable excipients selected by one of skill in the art. Different solid forms can be used including films, powders, granules or tablets. Any convenient excipient can be included within the composition and/or the solid form. Examples include, but are not limited to, viscosity-building agents (both polymeric and nonpolymeric), hydrophilicity agents (both polymeric and nonpolymeric), binders, coloring agents and other excipients described herein. The solid form can be prepared by forming a film that contains the active ingredient(s) and excipients. The film can then be divided into discrete units that contain an efficacious amount of the active component. Alternatively, the solid form of the composition can be prepared by compression of a powder mixture using procedures like those used to prepare pharmaceutical tablets. Other solid forms of the composition suitable for application to the water-soluble, bioerodible pharmaceutical device can be devised by one of skill in the art.

The composition of the solid form of the composition used can vary and depends upon the active ingredient(s) or pharmaceutical(s) employed as well as the other components of the composition. In general, one of skill in the art can select suitable and appropriate excipients for the solid form of the composition to be incorporated into the water-soluble carrier device.

The active ingredient(s) or pharmaceutical(s) of interest should be present at a quantifiable concentration in the liquid, solid, molten or powder form of the composition so that a therapeutically effective dosage can be calculated and dispensed or applied with precision to the water-soluble, bioerodible pharmaceutical device.

If applied as a liquid form such as a solution or suspension, the solution or suspension is preferably sufficiently concentrated so that a fairly small aliquot will contain a therapeutically effective amount of the active ingredient(s) or pharmaceutical(s) of interest. Similarly, if applied as a solid form, such as a discrete film or tablet, the discrete film or tablet is preferably sufficiently concentrated so that a fairly small discrete film or tablet will contain a therapeutically effective amount of the active ingredient(s) or pharmaceutical(s) of interest when compared to the size of the water-soluble, bioerodible pharmaceutical device.

The water-soluble, bioerodible pharmaceutical device employed in the invention has a water-soluble bioadhesive layer that is applied to the surface of a mucosal membrane of a mammal. The device may also have a water-soluble non-bioadhesive backing layer that protects the interior, bioadhesive layer. The water-soluble non-bioadhesive backing layer will dissolve first. One example of such a device is the BEMA™ delivery system produced by Atrix Laboratories, Inc.

An aliquot of the liquid form or discrete unit of pharmaceutical composition that contains a therapeutically effective amount of the active ingredient(s) or pharmaceutical(s) is applied directly onto the selected surface, preferably the surface of the bioadhesive layer, of the pre-assembled water-soluble, bioerodible pharmaceutical device. Any suitable dispensing equipment can be used for applying the pharmaceutical composition solution to the selected surface. If the liquid form of the pharmaceutical composition is employed, examples of micro-dispensing applicators that can be used to dispense aliquots include the IVEK® Precision Liquid Metering System. The aliquot is dried or otherwise stably adsorbed onto the surface of the selected surface to form an active ingredient-containing or pharmaceutical-containing deposit on the surface of the selected layer. If the aliquot is to be dried, drying of the dispensed solution is by any convenient means known to be acceptable for film drying. Examples of convenient drying methods include air-drying at ambient conditions or drying in a conventional film-drying oven.

However, the drying step is sometimes performed to facilitate handling, packaging and easy administration of the composition. Many liquid pharmaceutical compositions may be therapeutically effective and/or commercially acceptable without drying. In such a case, the liquid form of the pharmaceutical composition can be applied to the water-soluble, bioerodible pharmaceutical device immediately prior to application to the surface of a mucosal membrane of a mammal. If the solid form of the pharmaceutical composition is employed, the discrete film or tablet is placed directly onto the selected surface of the pre-assembled water-soluble, bioerodible pharmaceutical device. Any suitable dispensing equipment can be used for placing the discrete film or tablet to the chosen surface.

If being placed on the bioadhesive surface, the discrete film or tablet can be laminated to the bioadhesive surface to securely bond the discrete film or tablet and bioadhesive surface together. Alternatively, a small aliquot of suitable liquid such as water can be used to wet the bioadhesive surface to temporarily increase its adhesiveness and allow the discrete film or tablet to bond securely to its surface. Other methods of bonding the discrete film or tablet to the bioadhesive surface can be devised by one of skill in the art.

After application and/or adsorption of the pharmaceutical composition, the water-soluble, bioerodible pharmaceutical device can be packaged for sale and/or used for administration of the active ingredient(s) or pharmaceutical(s) in the composition deposited onto the surface of the device.

Drug Delivery Devices

The present invention relates to applying or depositing a composition containing an active ingredient(s) or pharmaceutical(s) to a water-soluble carrier device that is used for sustained delivery of the composition after application of the device to a mucosal surface. Such drug delivery devices protect and deliver active ingredient(s) or pharmaceutical(s) to the site of application, to surrounding tissues, and to bodily fluids in the area of application, and/or provide systemic delivery. Such drug delivery devices desirably have an effective residence time, cause minimal discomfort and are easy to use.

In one embodiment, the present methods are used to deposit a composition to a mucoadhesive film having two layers-a bioadhesive layer and a non-bioadhesive backing layer. Both the bioadhesive layer and the non-bioadhesive backing layer are water-soluble and are both made of materials recognized or established as safe for human or animal use. The pharmaceutical composition is generally applied to the surface of the bioadhesive layer, which is closest to the application site. The backing layer protects the interior, bioadhesive layer and will dissolve first. Dissolution of the backing layer primarily controls the residence time of the film disc after application to the mucosal surface.

The bioadhesive layer may comprise at least one film-forming water-soluble polymer (the “film-forming polymer”) and at least one pharmacologically acceptable polymer known for its bioadhesive capabilities (the “bioadhesive polymer”). Alternatively, the adhesive composition for the bioadhesive layer contains at least a water-soluble polymer and a water-soluble plasticizer such as glycerin.

The film-forming polymer of the bioadhesive layer can be a cellulose derivative. Such a film-forming polymer may comprise hydroxyethyl cellulose, hydroxypropyl cellulose, hydroxypropylmethyl cellulose, hydroxyethylmethyl cellulose, or a combination thereof. Similar film-forming polymers may also be used. The film-forming polymer may be crosslinked or plasticized in order to alter its dissolution kinetics.

The bioadhesive polymer of the bioadhesive layer may comprise polyacrylic acid (PM), which may or may not be partially crosslinked, sodium carboxymethyl cellulose (NaCMC), or polyvinylpyrrolidone (PVP), or combinations thereof. These bioadhesive polymers are preferred because they have good and instantaneous mucoadhesive properties in a dry, film state. Other bioadhesive polymers having similarly useful properties and that known to one of skill in the art may also be used.

The simultaneous use of PM with some grades of PVP may result in the precipitation of one or both components. This precipitation may not be desirable, especially when attempting to form a homogenous layer. Moreover, such precipitation may slightly alter the overall adhesive properties of the device. One of skill in the art can recognize these problems and avoid use of those grades of PVP with PM.

The non-bioadhesive backing layer may comprise a water-soluble, film-forming pharmaceutically acceptable polymer such as, but not limited to, hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, or a combination thereof. The backing layer component may or may not be crosslinked. In one embodiment, the backing layer component comprises hydroxyethyl cellulose and hydroxypropyl cellulose.

Combinations of different polymers or similar polymers with definite molecular weight characteristics may be used in order to achieve preferred film forming capabilities, mechanical properties, and kinetics of dissolution.

In order to modify the water dissolution kinetics of the backing layer without resulting in a non-water soluble material, partial and limited crosslinking may be used. Crosslinking agents known in the art are appropriate for use in the invention and may include glyoxal, propylene glycol, glycerol, dihydroxy-polyethylene glycol of different sizes, and butylene glycol. The amount of crosslinking agent used may vary, depending on the particular polymers and crosslinking agent, but should not exceed 5% molar equivalent of the polymeric material, and preferably comprises 0 to 3% molar equivalent of the polymeric material. Dissolution characteristics may be adjusted to modify the residence time and the release profile of a drug when included in the backing layer.

The thickness of the device may vary, depending on the thickness of each of the layers. Preferably, the bilayer thickness ranges from 0.05 mm to 1 mm, and more preferably from 0.1 to 0.5 mm. The thickness of each layer may vary from 10 to 90% of the overall thickness of the bilayer device, and preferably varies from 30 to 60%. Thus, the preferred thickness of each layer may vary from 0.01 mm to 0.9 mm, and more preferably from 0.03 to 0.6 mm.

The water-soluble, bioerodible pharmaceutical device may be prepared by numerous methods known in the art. In one embodiment, the components of the separate layers are separately dissolved in the appropriate solvent or combination of solvents to prepare a solution or suspension suitable for coating. Solvents for use in the present invention may comprise water, methanol, ethanol, or low alkyl alcohols such as isopropyl alcohol, acetone, methyl ethyl acetone, heptane, or dichloroethane, alone or combination. The final solvent content or residual solvent content in the film may be the result of either or both layers. The solvent may also be used as a plasticizer or dissolution-rate-modifying agent. Solvents having less volatility such as glycerin, propylene glycol, and polyethylene glycol may be part of the composition to plasticize the final device.

The bioadhesive or backing solutions are then separately coated onto an appropriate manufacturing substrate. Each solution is cast and processed into a thin film by techniques known in the art, such as by film dipping, film coating, film casting, spin coating, or spray drying using the appropriate substrate. The thin film is then dried. The drying step can be accomplished in any type of oven. However, the drying procedure should be selected to be compatible with the solvent employed and the amount of residual solvent may depend on the drying procedure. One of skill in the art can readily select appropriate drying procedures for the selected solvent(s). The film layers may be prepared independently and then laminated together or may be prepared as films, one on the top of the other.

The combined film obtained after the layers have been laminated together, or coated on top of each other, may be cut into any type of shape, for application to the mucosal tissue. Some shapes include discs, ellipses, squares, rectangles, and parallelepipeds.

Post-Loading of the Pharmaceutical

To load the pre-formed, water-soluble, bioerodible pharmaceutical device with a composition containing active ingredient(s) or pharmaceutical(s), a liquid form of the composition is prepared. The liquid form can be a solution or suspension that contains the composition of active ingredient(s) or pharmaceutical(s) and any pharmaceutically acceptable excipients selected by one of skill in the art. Alternatively, a solid form of the composition such as a discrete film or tablet is prepared that contains the active ingredient(s) or pharmaceutical(s) and any pharmaceutically acceptable excipients selected by one of skill in the art.

The active ingredient(s) or pharmaceutical(s) of interest should be present at a quantifiable concentration so that a therapeutically effective dosage can be calculated and dispensed or applied with precision. The liquid or solid forms of the pharmaceutical composition is preferably sufficiently concentrated so that a fairly small aliquot or discrete unit will contain a therapeutically effective amount of the active ingredient(s) or pharmaceutical(s) of interest.

Pharmaceutical compositions used in the methods and devices of the invention may comprise a single pharmaceutical or a combination of pharmaceuticals. Examples of categories of pharmaceuticals that may be used, either alone or in combination include: andrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunt to; anesthetic; anorectic; antagonist; anterior pituitary suppressant; anthelmintic; antiacne agent; antiadrenergic; anti-allergic; anti-amebic; anti-androgen; anti-anemic antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infctive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enxzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregaton inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; zxanthine oxidase inhibitor.

Active ingredients or pharmaceuticals that are examples of these categories include, but are not limied to, Acebutolol; Acebutolol; Acyclovir; Albuterol; Alfentanil; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; Zolpidem.

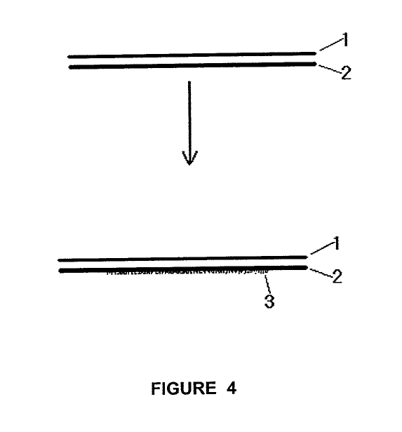

FIG. 4 illustrates how a bioerodible, water-soluble carrier device is post-loaded. The bioerodible, water-soluble carrier device is pre-formed to have a non-bioadhesive backing layer 1 and a bioadhesive layer 2. To post-load onto a surface of the bioadhesive layer 2 of such a pre-formed device, the composition is incorporated onto the bioadhesive layer 2. Alternatively, the composition can be incorporated onto the surface of the non-bioadhesive backing layer 1. After incorporation of the composition, the loaded bioerodible, water-soluble, carrier device has a composition deposit or layer 3, for example, on the bioadhesive layer 2. While the composition deposit or layer 3 can extend to the periphery of the bioadhesive layer 2, it preferably covers less than the total surface of the bioadhesive layer 2, and is present near the center of the layer, so that upon application of a device, a portion of the bioadhesive layer 2 will adhere to the mucosal surface all around the composition deposit or layer 3.

As used herein, a “loaded” device is a water-soluble, bioerodible pharmaceutical device that has a selected composition incorporated onto or into the device. Also, as used herein, “loading” generally means incorporating a selected composition onto or into such a device. While post-loading generally places the composition onto a selected surface of the device, some diffusion of the composition into the layer, or into more than one layer, of the device may occur. Hence, while most of the composition may be on a surface of a post-loaded device, depending upon the solvent or components of the selected composition(s) and surface(s), diffusion may occur and the composition may be present within one or more layers of the device. Such diffusion will not adversely affect the properties of the device. One of skill in the art can readily observe the pharmacokinetic properties of the device and modify those properties as needed.

The amount of active ingredient(s) or pharmaceutical(s) to be placed in the liquid, solid, molten or powder forms of the composition depends on the desired treatment dosage to be administered, although preferably, the active, pharmaceutical component comprises 0.001 to 50% by weight of the water-soluble, bioerodible pharmaceutical device, and more preferably between 0.005 and 35% by weight.

The composition(s) may be dispensed or applied just once to a surface of the device, or as multiple discrete deposits onto the surface of the selected layer(s). The application of multiple deposits onto the surface of a selected layer(s) is done for various reasons. Such reasons include, but are not limited to increasing the surface area of drug absorption, to conferring production advantages such as reduced drying times, to allowing for deposits of different compositions to be applied to the same drug delivery device, and/or to separating different active pharmaceutical(s) into distinct deposits. Increasing the surface area of drug absorption results in faster absorption of active ingredient(s) or pharmaceutical(s) and reduces the time required to achieve efficacious concentrations of drug in the blood.

By applying different compositions of the active ingredient(s) or pharmaceutical(s), changes in absorption kinetics can be produced from each distinct deposit and result in pharmacokinetic profiles that are a composite of the profiles obtained from each distinct deposit. This manipulation of the pharmaceutical compositions can be used to achieve, for example, sustained blood levels of the active pharmaceutical(s).

Applying distinct deposits of different active pharmaceuticals will result in combination drug delivery devices. Application of distinct deposits may be required for certain combinations of active pharmaceuticals due to their chemical incompatibility if contained in the same pharmaceutical composition. In addition, different pharmacokinetic profiles may be desired for each active ingredient or pharmaceutical. Such different profiles may be achieved by incorporating each active ingredient or pharmaceutical into different compositions.

Any convenient excipient can be included in the composition to be applied to the surface of the water-soluble, bioerodibe pharmaceutical device. Polymeric and nonpolymeric viscosity-building agents can be included as excipients. Polymeric and nonpolymeric hydrophilicity agents can also be included as excipients. Pharmaceutically acceptable plasticizers, flavoring and coloring agents, and preservatives may also be included in the pharmaceutical composition. Preferably, these components comprise no more than 1% of the final weight of the device, but the amount may vary depending on the active ingredients, pharmaceuticals or other components of the device. One of skill in the art can readily achieve appropriate concentrations of these components.

If the liquid form of the composition is employed, the solvent used to dissolve or suspend the active ingredient(s) or pharmaceutical(s) can vary and depends upon the active ingredient(s) or pharmaceutical(s) employed as well as the other components of the pharmaceutical composition. In general, one of skill in the art can select a good solvent for the active ingredient(s) or pharmaceutical(s) to be incorporated into the water-soluble, bioerodible pharmaceutical device.

While some water can be present in the solvent, most solvents used for incorporating the active ingredient(s) or pharmaceutical(s) onto the device will usually contain little water. Water is generally avoided as the primary liquid solvent to prevent solubilization of the water-soluble components of the device during incorporation of the composition. Water is also avoided because it dries so slowly. A water-based composition may disperse over a larger area of the device, or even to run off of the device during loading. Some solvents, such as certain alcohols, contain small amounts of water. Such small amounts of water in the solvent are not problematic. However, large amounts of water, or use of water as the primary solvent for the composition, may lead to difficulties during loading.

Preferred solvents for the composition include organic-based solvents that have a high vapor pressure or a low normal boiling point and that have regulatory acceptance as a pharmaceutical solvent suitable for oral administration. Examples of solvents that may be used include ethanol or isopropanol.

An aliquot of the composition solution that contains a therapeutically effective amount of the active ingredient(s) or pharmaceutical(s) is applied directly onto the chosen surface of the pre-assembled water-soluble, bioerodible pharmaceutical device. Preferably, the surface is the surface of the bioadhesive layer. Dispensing equipment can be used for applying the pharmaceutical composition solution to the selected surface. Examples of microdispensing applicators that can be used include the IVEK® Precision Liquid Metering System. However, any suitable dispensing equipment can be employed. Examples of such dispensing equipment include precision syringes, pipetting equipment, and electronic fluid dispensers.

The aliquot is dried or otherwise stably adsorbed onto the surface of the selected surface to form an active- or pharmaceutical-containing deposit on the surface of the device. Drying of the dispensed solution is by any convenient means known to be acceptable for film drying. Examples of convenient drying methods include drying at ambient conditions or in a conventional film-drying oven. Alternatively, it may be desired for specific product characteristics to maintain the aliquot as a deposit liquid.

A molten composition is prepared by mixing the active ingredient(s) or pharmaceutical(s) with the selected excipients, melting the composition and dispensing the melted composition onto a surface of the device. As for the other compositions, any desirable excipient can be included. However, some attention is paid to whether the selected excipients will melt at a temperature that is convenient for dispensing the composition onto the device. Similarly, each active ingredient, pharmaceutical and excipient should be stable at the temperatures used for melting and dispensing the composition.

If a solid or powder form of the composition is employed, the formulation of the pharmaceutical composition used to contain the active ingredient(s) or pharmaceutical(s) can vary and depends upon the active ingredient(s) or pharmaceutical(s) employed as well as the other components of the composition. In general, one of skill in the art can select suitable excipients for the active ingredient(s) or pharmaceutical(s) of interest to be incorporated into the water-soluble, bioerodible pharmaceutical device. Preferred excipients include, but are not limited to, components similar to those included in the bioadhesive composition. Examples include, but are not limited to, viscosity-building agents (both polymeric and nonpolymeric), hydrophilicity agents (both polymeric and nonpolymeric), binders, coloring agents and other excipients described herein. Other examples of excipients that may be used include polyacrylic acid (PAA), which may or may not be partially crosslinked, sodium carboxymethyl cellulose (NaCMC), or polyvinylpyrrolidone (PVP), or combinations thereof.

When the composition is to be deposited in a solid form, different solid forms can be used including films, powders, granules or tablets. Any convenient excipient can be included within the composition and/or the solid form. The solid form can be prepared by forming a film that contains the active ingredient(s) and excipients. The film comprises water-soluble polymers known to those of skill in the art, for example, some of the water-soluble polymers described herein. Each film can be prepared as a discrete unit, or the film can be divided into discrete units from a larger film, so that the individual films contain an efficacious amount of the active component. Alternatively, the solid form of the composition can be prepared by compression of a powder mixture using procedures like those used to prepare pharmaceutical tablets. Other solid forms of the composition suitable for application to the water-soluble, bioerodible pharmaceutical device can be devised by one of skill in the art.

A discrete film or tablet containing the composition with the desired dosage or amount of the active ingredient(s) or pharmaceutical(s) is applied directly onto the chosen surface of the pre-assembled water-soluble, bioerodible pharmaceutical device. The desired amount applied is a therapeutically effective amount, but that amount can be achieved by multiple, discrete applications of separate films, tablets, powders or other solid forms. Application equipment can be used for placing the discrete film(s) or tablet(s) or other solid forms onto the selected surface. The discrete film or tablet is preferably securely bonded onto the surface of the bioadhesive layer to form an active pharmaceutical-containing deposit on the surface of the bioadhesive layer. Bonding the discrete film or tablet is by any convenient means known to be acceptable for bonding films together drying. Examples of convenient bonding methods include lamination or surface hydration.

After application and drying/adsorption of the composition to form a stable deposit, the water-soluble, bioerodible pharmaceutical device can be packaged for sale and/or used for administration of the active ingredient(s) or pharmaceutical(s) in the composition deposited onto the selected surface of the device.

Desirable Uses of the Drug Delivery System

Water-soluble, bioerodible pharmaceutical devices formed by the methods of the invention can be used in the localized treatment of body tissues, diseases, or wounds that may have moist surfaces and that are susceptible to bodily fluids, such as the mouth, the vagina, or other types of mucosal surfaces. Water-soluble, bioerodible pharmaceutical devices formed by the methods of the invention can also be used for the systemic delivery of active pharmaceutical(s) through body tissues, diseased tissue, or wounds that may have moist surfaces and that are susceptible to bodily fluids, such as the mouth, the vagina, or other types of mucosal surfaces. These moist surfaces include, but are not limited to, the mouth, the vagina and other mucosal or epithelial covered tissues. The device carries active ingredient(s) or pharmaceutical(s), and upon application and adherence to the mucosal surface, offers a layer of protection and delivers the active ingredient(s) or pharmaceutical(s) to the application site, the surrounding tissues, and other bodily fluids. The device provides an appropriate residence time for effective drug delivery at the application site, given the control of solubilization in aqueous solution or bodily fluids such as saliva, and the slow, natural dissolution of the film concomitant to the delivery.

Devices made by the methods of the invention offer the advantages of an effective residence time with minimal discomfort and ease of use, and are an appropriate vehicle for the local as well as systemic delivery of active ingredient(s) or pharmaceutical(s), given its thinner, flexible form.

Devices formed by the methods of the invention are made of water-soluble components and are bioerodible. The use of water-soluble components allows the device to dissolve over a period of time, with natural bodily fluids slowly dissolving and eroding away the carrier, while the active ingredient(s) or pharmaceutical(s) remains at the application site. Unlike bandages, transdermal devices and other non-water-soluble film systems, the user of the present invention does not have to remove the device following treatment. Nor does the user experience the sensation of the presence of a foreign object at the mucosal surface or within the body cavity, given that upon application, water absorption softens the device, and over time, the device slowly dissolves or erodes away.

The residence times of water-soluble, bioerodible pharmaceutical devices made by the methods of the invention depend on the dissolution rate of the water-soluble polymers used. Dissolution rates may be adjusted by mixing together chemically different polymers, such as hydroxyethyl cellulose and hydroxypropyl cellulose; by using different molecular weight grades of the same polymer, such as mixing low and medium molecular weight hydroxyethyl cellulose; by using crosslinking agents such as glyoxal with polymers such as hydroxyethyl cellulose for partial crosslinking; or by post-treatment irradiation or curing, that may alter the physical state of the film, including its crystallinity or phase transition, once obtained. These strategies might be employed alone or in combination in order to modify the dissolution kinetics of the device, without suppressing the water solubility characteristics of the component materials.

Upon application, the pharmaceutical delivery device adheres to the mucosal surface and remains in place. Water absorption softens the device quickly, diminishing and eliminating the foreign body sensation. As the device rests upon the mucosal surface, delivery of the active ingredient(s) or pharmaceutical(s) is provided. Residence times may vary, depending on the formulation and materials used, but may be modulated between a few minutes to several hours.

The examples are intended to further illustrate, but not limit, the invention. These examples illustrate postloading methods that overcome incompatibilities between actives and ingredients of the mixtures processed to form film layers. The following examples also illustrate how post-loading reduces the amount of scrap generated during cutting of the film devices. The ability of post loaded bioerodible mucoadhesive devices to systemically deliver drugs is illustrated and the unexpected result that postloaded devices can provide significantly improved drug delivery compared to otherwise equivalent preloaded film is demonstrated.

Those skilled in the art will recognize that, while specific embodiments have been illustrated and described, various modifications and changes may be made without departing from the spirit and scope of the invention.

### EXAMPLE 1

Fifteen kilograms of backing solution was made by combining on a weight/weight basis 77.0% purified water, 0.46% sodium benzoate, 0.46% titanium dioxide, 9.92% hydroxyethyl cellulose, 9.92% hyrdoxypropyl cellulose, 0.23% tocopherol acetate, 0.02% citric acid, 0.05% methyl paraben, 0.02% propyl paraben, 0.23% saccharin, and 1.69 sweet peppermint. Mixing was maintained until batch was homogeneous.

Fifteen kilograms of bioadhesive solution was made by combining on a weight/weight basis 91.0% purified water, 1.55% polyacrylic acid, 0.06% FD & C yellow #5 lake, 2.21% hyroxyethyl cellulose, 0.33% hyrdoxypropyl cellulose, 0.09% tocopherol acetate, 0.01% citric acid, 0.02% methyl paraben, 0.01% propyl paraben, 4.72% sodium carboxymethyl cellulose.

Using a Werner Mathis web coater, the backing solution was coated upon a polyester substrate. The solution was coated under a knife with a wet gap of 0.90 mm. The coated film was then dried at 90° C. for 11 minutes. The bioadhesive solution was then coated upon the dried backing film under a knife with a wet gap of 1.37 mm. The film was dried at 90° C. for 11 minutes.

The bilayer film formed from sequential coating of the backing and bioadhesive solutions produced a bioerodible, water-soluble carrier device suitable for combination with active ingredients such as flavors, nutriceuticals, or pharmaceuticals.

### EXAMPLE 2

For a given 10″×10″ section of film of example 1, 9/16″ diameter discs of fentanyl citrate preloaded in the bioadhesive layer and cut approximately 1¼″ apart on center would result in 64 product discs. The calculated area of the discs is approximately 16 in², resulting in a scrap amount of 84%. The disc spacing was chosen for packaging considerations.

### EXAMPLE 3

For a given 10″×10″ section of film of example 1, ⅝″ diameter discs of hydrocodone preloaded in the bioadhesive layer and cut approximately 1¼″ apart on center would result in 64 product discs. The calculated area of the discs is approximately 20 in², resulting in a scrap amount of 80%. The disc spacing was chosen for packaging considerations.

### EXAMPLE 4

A 40 mL post-loading solution was made using 2.5% by weight fentanyl citrate, 79.95% by weight methanol, 14.625% by weight polyethylene glycol, and 2.925% by weight hydroxypropyl cellulose.

This solution was used for post-load dispensing. Post-loading was accomplished by a microdispensing applicator and the post-load dispense weights were verified by microbalance. A single drop of 12.6 mg of solution was dispensed on a single BEMA disc. The solvent was driven off at an elevated temperature and then allowed to cool to room temperature.

Using the same calculation procedure as example 2, the scrap amount for post-loaded fentanyl discs is 0% compared to the 84% scrap rate calculated for preloaded fentanyl discs.

### EXAMPLE 5

A 40 mL post-loading solution was made using 20.00% by weight hydrocodone free base, 65.12% by weight ethanol, 6.00% by weight glacial acetic acid, 7.40% by weight polyethylene glycol, and 1.48% by weight hydroxypropyl cellulose.

This solution was used for post-load dispensing. Post-loading was accomplished by a microdispensing applicator and the post-load dispense weights were verified by microbalance. Three (3) distinct drops of 5 mg of solution were dispensed onto a single BEMA disc. The solvent was driven off at an elevated temperature and then allowed to cool to room temperature.

Using the same calculation procedure as example 3, the scrap amount for post-loaded hydrocodone discs is 0% compared to the 80% scrap rate calculated for preloaded hydrocodone discs.

### EXAMPLE 6

A stock formulation was made by dispersing 2.87 grams of the opiod analgesic into 10.55 grams of bioadhesive solution. A low-sag sol with density of approximately 1.058 g/ml, and containing 51% (dry weight) of evenly dispersed active was obtained. This sol was applied to a casting sheet (0.005″ gauge silicone-coated film, grade 10668; Loparex, Inc.) previously installed as a substrate on the Werner-Mathis Labcoater. The Knife-over-Roll was adjusted to 0.051 mm gap over the substrate film, then the coating gap was set to 1.38 mm. The sol was coated by slowly drawing down the knife-edge over the sol, then drying in the coater-oven at 80° C. for 10 minutes. The coated film had a measured thickness is 0.8 mm. Circular discs of 0.25″ diameter were die-cut from the dried film. Average disc weights (N=6) were 15.3 mg. The amount of active in each solid disc was 8 mg.

A bilayer film was prepared as described in Example 1, and circular discs of ⅝″ diameter were die-cut from the film. This pre-assembled water-soluble, bioerodible pharmaceutical device was moistened with 5 μl of distilled water. A 0.25″ diameter circular disc of the discrete solid film described above was laminated onto the bioerodible device. The solid postload was bonded to the bioerodible pharmaceutical device. The final product was a bilayer disc having a discrete solid 0.25″ diameter postload containing approximately 10 mg of active.

### EXAMPLE 7

A 100 ml solution of placebo bioadhesive was made with 91% (w/w %) water, 2% (w/w%) hydroxyethyl cellulose, 0.3% (w/w%) hydroxypropyl cellulose, 1.5% (w/w%) polyacrylic acid, 5% (w/w%) sodium carboxymethyl cellulose, and 0.2% (w/w%) of a blend of red lake #40, methylparaben, propylparaben, tocopheryl acetate, citric acid, and glycerol. A 25 ml aliquot of this solution was removed and ondansetron base was added at a concentration of 4.33% by weight. Using a Werner Mathis Labcoater, the substrate (Mylar 1000D or other polyester films such as 3M ScotchPak 1022) was secured, and the backing layer solution was set in front of a knife over-roll with an opening of 1.0 mm. The backing solution was coated and the film dried for 12 minutes at 90° C. The pre-loaded adhesive was coated over the dried backer film with a knife height of 1.40 mm and dried for 12 minutes at 90° C. Discs were cut to ⅝ inch diameter so that each disc contained a target of 8 mg ondansetron base. The resulting product is an example of a preloaded water-soluble, bioerodible pharmaceutical device.

### EXAMPLE 8

A 100 ml solution of placebo bioadhesive was made with 91% (w/w %) water, 2% (w/w%) hydroxyethyl cellulose, 0.3% (w/w%) hydroxypropyl cellulose, 1.4% (w/w%) polyacrylic acid, 5.1% (w/w%) sodium carboxymethyl cellulose/sodium hydroxide blend and 0.2% of a blend of red lake #40, methylparaben, propylparaben, tocopheryl acetate, citric acid, and glycerol. Using a Werner Mathis Labcoater, the substrate (Mylar 1000D or other polyester films such as 3M ScotchPak 1022) was secured, and the backing layer solution was set in front of a knife over-roll with an opening of 1.0 mm. The backing solution was then cast and the film dried for 12 min. at 90° C. The placebo adhesive was cast over the dried backer film with a knife height of 1.25 mm and dried for 12 minutes at 90° C. Discs were cut to ⅝ in. diameter. A solution was prepared with 28.5% ondansetron base in glacial acetic acid. This solution was applied to the BEMA™ placebo discs using an electronic fluid dispenser (EFD, XL1000) set to dispense approximately 27 mg of this solution, so that each disc contained a target 8 mg ondansetron base. The resulting product is an example of a post-loaded water-soluble, bioerodible pharmaceutical device.

### EXAMPLE 9

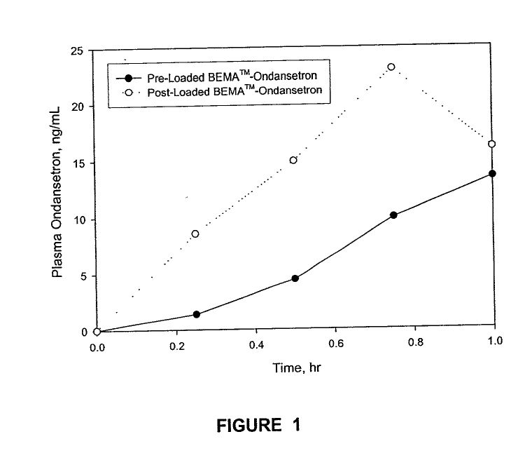

Two BEMA™-Ondansetron test articles yielded significantly different pharmacokinetic profiles when administered to five dogs. A pre-loaded BEMA™-Ondansetron disc was prepared as in Example 7 and a postloaded BEMA™-Ondansetron disc was prepared as in Example 8. FIG. 1 shows the mean pharmacokinetic profile for the pre-loaded and post-loaded BEMA™-Ondansetron discs at the initial time points (less than 1 hour). In general, the post-loaded test articles led to more rapid absorption of ondansetron upon buccal administration. Specifically, the maximum mean plasma ondansetron concentration for the post-loaded discs, Cₘₐₓ=23.0+6.06 ng/mL, occurred at 45 minutes after administration compared to the Cₘₐₓ for the pre-loaded discs, 16.3±3.13 ng/mL, reached at 1.5 hr post-administration. The rapid uptake of ondansetron from the post-loaded discs also led to a significantly higher (p<0.05) mean area-under-the-curve (AUC). The AUC for the post-loaded discs was 87.8±10.3 hr·ng/mL compared to 50.0±7.07 hr·ng/mL for the pre-loaded BEMA™-Ondansetron discs.

### EXAMPLE 10

A bioadhesive solution and a backing solution functionally equivalent to those described in example 1 were prepared. Sufficient hydrocodone bitartrate was added to the bioadhesive mixture to form a 3.0% (w/w%) solution of drug.

Using a Werner Mathis Labcoater, the substrate (Mylar 1000D or other polyester films such as 3M ScotchPak 1022) was secured, and the backing layer solution was set in front of a knife-over-roll with an opening of 0.70 mm from the surface of the substrate. The backing solution was then cast and the film dried for 15 minutes at 80° C. The knife was raised to 0.80 mm from the surface of the substrate, and a second coating of backer solution was applied. The film dried for 15 minutes at 80° C. The knife was raised to 1.10 mm from the surface of the substrate and a layer of bioadhesive was coated onto the backer. The film was dried for 15 minutes at 60° C. A second coating of bioadhesive was applied at the same coating conditions. The film was dried for 30 minutes at 60° C. Discs were cut to a ⅝ inch diameter. Hydrocodone free base equivalent concentration in the discs were 5 mg. This is an example of a preloaded BEMA™-Hydrocodone formulation.

### EXAMPLE 11

A bioadhesive solution and a backing solution functionally equivalent to those described in example 1 were prepared. Using a Werner Mathis Labcoater, the substrate (Mylar 1000D or other polyester films such as 3M ScotchPak 1022) was secured, and the backing layer solution was set in front of a knife over-roll with an opening of 0.90 mm from the surface of the substrate. The backing solution was then cast and the film dried for 10 minutes at 90° C. The knife was raised to 1.25 mm from the surface of the substrate and a layer of bioadhesive was coated onto the backer and dried for 10 minutes at 90° C. Discs were cut to ⅝ in. diameter. A solution was prepared with 20% hydrocodone free base, 65.12% (w/w%) ethanol (190 Proof), 6.0% (w/w%) glacial acetic acid, 1.48% (w/w%) hydroxypropyl cellulose, and 7.4% polyethylene glycol 3350. This solution was applied to the BEMA™ placebo discs using an electronic fluid dispenser (EFD, XL1000) set to dispense approximately 5 mg of this solution. Three discrete deposits were formed resulting in each disc containing 3 mg hydrocodone. This is an example of a post-loaded BEMA™-Hydrocodone formulation.

### EXAMPLE 12

Two different formulations of BEMA™-Hydrocodone test articles yielded significantly different pharmacokinetic profiles when each was administered to five dogs. A preloaded BEMA™-Hydrocodone disc was prepared as in Example 10, and a post-loaded BEMA™-Hydrocodone disc with three discrete post-load deposits was prepared as in Example 11.

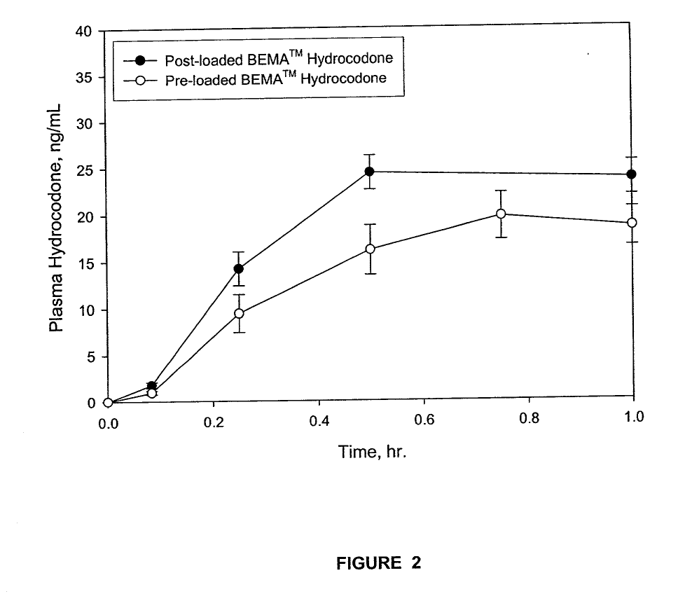

FIG. 2 shows the mean pharmacokinetic profile for the preloaded and post-loaded BEMA™-Hydrocodone discs. The preloaded BEMA™-Hydrocodone formulation contained approximately 67% more hydrocodone than the post-loaded BEMA™-Hydrocodone formulation. The mean plasma hydrocodone concentration was observed for the preloaded BEMA™ and a maximum concentration, Cₘₐₓ, of 20.9±3.8 ng/mL was observed at tₘₐₓ 1.25 hr. In contrast, the post-loaded discs gave rise to a higher Cₘₐₓ of 24.4±1.8 ng/mL in less time (tₘₐₓ=30 min). In general, the area-under-the-curve (AUC) for the post-loaded disc formulation with three discrete deposits was AUC=58.6 hr·ng/mL compared to an AUC=60.8 hr·ng/mL for the preloaded disc formulation. Therefore, the post-loaded test articles with three discrete deposits provided enhanced bioavailability, gave rise to more rapid absorption of hydrocodone upon buccal administration, and showed less variability in pharmacokinetic profile than the preloaded disc formulation.

### EXAMPLE 13

A bioadhesive solution and a backing solution functionally equivalent to those described in example 1 were prepared.

Using a Werner Mathis Labcoater, the substrate (Mylar 1000D or other polyester films such as 3M ScotchPak 1022) was secured, and the backing layer solution was set in front of a knife over-roll with an opening of 0.75 mm from the surface of the substrate. The backing solution was then cast and the film dried for 12 minutes at 90° C. The knife was raised to 1.45 mm from the surface of the substrate and a layer of bioadhesive was coated onto the backer and dried for 10 minutes at 90° C. Discs were cut to ⅝ in. diameter. A solution was prepared with 15% (w/w%) hydrocodone free base, 38.2% (w/w%) methanol, 19.1% (w/w%) ethanol (190 Proof), 19.1% (w/w%) water, 10% (w/w%) glacial acetic acid, 4.5% (w/w%) hydroxypropyl cellulose, and 9% (w/w%) polyethylene glycol 3350. This solution was applied to the BEMA™ placebo discs using an electronic fluid dispenser (EFD, XL1000) set to dispense approximately 20 mg of this solution. One discrete deposit was formed resulting in each disc contained 3 mg hydrocodone. This is an example of a post-loaded BEMA™-Hydrocodone formulation.

### EXAMPLE 14

A bioadhesive solution and a backing solution functionally equivalent to those described in example 1 were prepared.

Using a Werner Mathis Labcoater, the substrate (Mylar 100OD or other polyester films such as 3M ScotchPak 1022) was secured, and the backing layer solution was set in front of a knife over-roll with an opening of 0.75 mm. The backing solution was then cast and the film dried for 12 minutes at 90° C. The knife was raised to 1.45 mm and a layer of bioadhesive was coated onto the backer and dried for 10 minutes at 90° C. Discs were cut to a ⅝ in. diameter. A solution was prepared with 15% hydrocodone free base, 38.2% (w/w%) methanol, 19.1% (w/w%) ethanol (190 Proof), 19.1% (w/w%) water, 10% (w/w%) glacial acetic acid, 4.5% (w/w%) hydroxypropyl cellulose, and 9% (w/w%) polyethylene glycol 3350. This solution was applied to the BEMA™ placebo discs using an electronic fluid dispenser (EFD, XL1000) set to dispense approximately 3 mg of this solution. Five discrete deposits were formed resulting in each disc contained 3 mg hydrocodone. This is an example of a post-loaded BEMA™-Hydrocodone formulation.

### EXAMPLE 15

Three different formulations of neutralized BEMA™-Hydrocodone test articles yielded significantly different pharmacokinetic profiles when each was administered to five dogs, even though the different formulations contained equivalent dosages of hydrocodone. A post-loaded BEMA™-Hydrocodone disc with one discrete post-load deposit was prepared as in Example 13, a post-loaded BEMA™-Hydrocodone disc with three discrete post-load deposits was prepared as in Example 11, and a post-loaded BEMA™-Hydrocodone disc with five discrete post-load deposits was prepared as in Example 14.

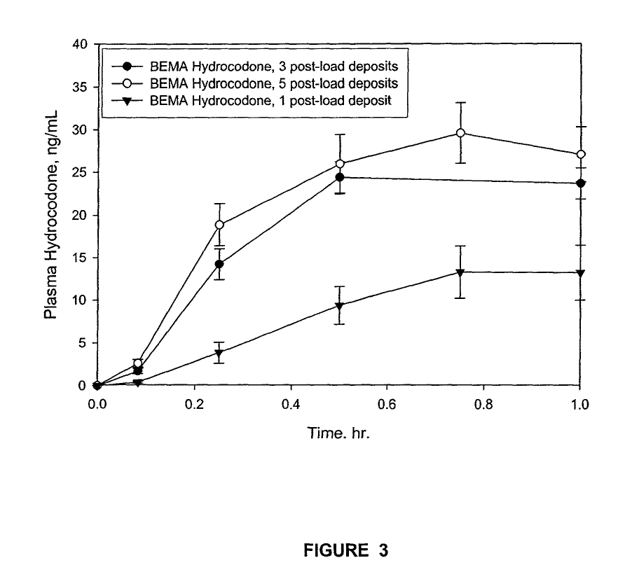

FIG. 3 shows the mean pharmacokinetic profile for the three different post-loaded BEMA™-Hydrocodone discs containing equivalent dosages of hydrocodone. Observation of the mean plasma hydrocodone concentration demonstrated that a maximum concentration (Cₘₐₓ) of 30 ng/mL occurred at 45 minutes for five discrete postload deposits compared to a Cₘₐₓ of 24 ng/mL at 30 minutes for three discrete postload deposits and a Cₘₐₓ of 13 ng/mL at 45 minutes for one discrete postload deposit. In general, area-under-the-curve (AUC) for the post-loaded discs with five discrete deposits was higher with an AUC 64.2 hr·ng/mL compared to the AUC for the post-loaded disc with three discrete deposits, AUC=58.6 hr·ng/mL and the AUC for the post-loaded disc with one discrete deposit, AUC=34.5 hr·ng/mL. In addition to enhanced bioavailability, the postloaded test articles with five discrete deposits had more rapid absorption of hydrocodone upon buccal administration.

What is claimed:

1. A bioerodible, water-soluble, carrier device comprising a non-bioadhesive backing layer, a bioadhesive layer and a composition comprising an active ingredient, wherein the composition is deposited onto a surface of either the non-bioadhesive backing layer or the bioadhesive layer after formation of the bioerodible, water-soluble, carrier device.

2. The bioerodible, water-soluble, carrier device of claim 1 wherein the composition does not cover the entire surface of the layer.

3. The bioerodible, water-soluble carrier device of claim 1 wherein the bioadhesive layer can adhere to a mucosal surface of a mammal.

4. The bioerodible, water-soluble carrier device of claim 1 wherein the composition forms a non-bioadhesive deposit after the composition is deposited onto a surface of either layer.

5. The bioerodible, water-soluble carrier device of claim 1 wherein the composition is deposited near the center of the surface of the bioadhesive layer and the periphery of the bioadhesive layer can adhere to a mucosal surface of a mammal.

6. The bioerodible, water-soluble carrier device of claim 1 wherein the composition further comprises a fluid carrier suitable for administration to a mucosal surface of a mammal.

7. The bioerodible, water-soluble carrier device of claim 6 wherein the fluid carrier comprises acetic acid, acetone, anisole, 1-butanol, 2-butanol, butyl acetate, tert-butylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, or tetrahydrofuran.

8. The bioerodible, water-soluble carrier device of claim 1 wherein the composition further comprises a viscosity-building agent.

9. The bioerodible, water-soluble carrier device of claim 1 wherein the composition further comprises a polymeric or nonpolymeric hydrophilicity agent.

10. The bioerodible, water-soluble carrier device of claim 9 wherein the hydrophilicity agent comprises polyethylene glycol.

11. The bioerodible, water-soluble carrier device of claim 1 wherein the bioadhesive layer is water-soluble.

12. The bioerodible, water-soluble carrier device of claim 1 wherein the bioadhesive layer comprises a film forming water-soluble polymer and a bioadhesive polymer.

13. The bioerodible, water-soluble carrier device of claim 12 wherein the film forming water soluble polymer of the bioadhesive layer comprises hydroxyethyl cellulose, hydroxypropyl cellulose, hydroxypropylmethyl cellulose, hydroxyethylmethyl cellulose, or a combination thereof.

14. The bioerodible, water-soluble carrier device of claim 12 wherein the film forming water soluble polymer of the bioadhesive layer is crosslinked or plasticized.

15. The bioerodible, water-soluble carrier device of claim 12 wherein the bioadhesive polymer of the bioadhesive layer comprises polyacrylic acid, sodium carboxymethyl cellulose or polyvinylpyrrolidone or a combination thereof.

16. The bioerodible, water-soluble carrier device of claim 15 wherein the polyacrylic acid is partially crosslinked.

17. The bioerodible, water-soluble carrier device of claim 1 wherein the non-bioadhesive backing layer comprises a pharmaceutically acceptable, film-forming, water-soluble polymer.

18. The bioerodible, water-soluble carrier device of claim 17 wherein the pharmaceutically acceptable, film-forming, water-soluble polymer is hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, or a combination thereof.

19. The bioerodible, water-soluble carrier device of claim 17 wherein the pharmaceutically acceptable, film-forming, water-soluble polymer comprises hydroxyethyl cellulose and hydroxypropyl cellulose.

20. The bioerodible, water-soluble carrier device of claim 17 wherein the pharmaceutically acceptable, film-forming, water-soluble polymer is crosslinked.

21. The bioerodible, water-soluble carrier device of claim 1 wherein the composition is a liquid composition when deposited onto a surface of either layer.

22. The bioerodible, water-soluble carrier device of claim 1 wherein the composition is a solid composition when deposited onto a surface of either layer.

23. The bioerodible, water-soluble carrier device of claim 1 wherein the composition is a molten composition when deposited onto a surface of either layer.

24. The bioerodible, water-soluble carrier device of claim 1 wherein the composition is deposited onto a surface of either layer more than once.

25. The bioerodible, water-soluble carrier device of claim 1 wherein the composition is deposited onto a surface of either layer between about 1 to about 10 times.

26. The bioerodible, water-soluble carrier device of claim 1 wherein the non-bioadhesive backing layer dissolves first.

27. The bioerodible, water-soluble carrier device of claim 1 wherein the device provides sustained delivery of the composition.

28. The bioerodible, water-soluble carrier device of claim 1 wherein the composition comprises an andrenergic agent; adrenocortical steroid; adrenocortical suppressant; alcohol deterrent; aldosterone antagonist; amino acid; ammonia detoxicant; anabolic; analeptic; analgesic; androgen; anesthesia; anesthetic; anorectic; antagonist; anterior pituitary suppressant; anthelmintic; anti-acne agent; antiadrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocin; plasminogen activator; platelet activating factor antagonist; platelet aggregaton inhibitor; post-stroke treatment agent; post-head trauma treatment agent; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; agent for treatment of amyotrophic laterial sclerosis; agent for treatment of cerebral ischemia; agent for treatment of Paget's disease; agent for treatment of unstable angina; uricosuric; vasoconstrictor; vasodilator; vulnerary; wound healing agent; or xanthine oxidase inhibitor.

29. The bioerodible, water-soluble carrier device of claim 1 wherein the composition comprises Acebutolol; Acebutolol; Acyclovir; Albuterol; Alfentanil; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; or Zolpidem.

30. The bioerodible, water-soluble carrier device of claim 1 wherein the composition comprises fentanyl.

31. The bioerodible, water-soluble carrier device of claim 1 wherein the composition comprises ondansetron.

32. The bioerodible, water-soluble carrier device of claim 1 wherein the composition comprises hydrocodone.

33. The bioerodible, water-soluble carrier device of claim 1 wherein the composition comprises between about 0.001 percent and about 50 percent by weight of the bioerodible, water-soluble, carrier device.

34. The bioerodible, water-soluble carrier device of claim 1 wherein the composition comprises between about 0.005 percent and about 35 percent by weight of the bioerodible, water-soluble, carrier device.

35. A method for incorporating a composition onto a preformed bioerodible, water-soluble carrier device comprising:
depositing the composition onto at least one surface of the preformed bioerodible, water-soluble carrier device to form a loaded bioerodible, water-soluble carrier device;
wherein the preformed bioerodible, water-soluble carrier device comprises at least one bioadhesive layer.

36. A method for incorporating a composition comprising at least one active ingredient and a fluid carrier onto a preformed bioerodible, water-soluble carrier device, the method comprising:
depositing at least one portion of the composition onto a surface of a layer of the preformed bioerodible, water-soluble carrier device to form a loaded bioerodible, water-soluble carrier device;
wherein the preformed bioerodible, water-soluble carrier device comprises at least one bioadhesive layer.

37. A method for incorporating at least one composition comprising at least one active ingredient and a solid carrier onto a preformed bioerodible, water-soluble carrier device comprising:
affixing at least one portion of the composition onto a surface of the preformed bioerodible, water-soluble carrier device to form a loaded bioerodible, water-soluble carrier device;
wherein the preformed bioerodible, water-soluble carrier device comprises at least one bioadhesive layer.

38. The method of claim 35, 36 or 37 wherein the preformed bioerodible, water-soluble carrier device further comprises a non-bioadhesive backing layer.

39. The method of claim 38 wherein the composition is deposited or affixed onto a surface of the non-bioadhesive backing layer.

40. The method of claim 35, 36 or 37 wherein the composition is deposited or affixed onto a surface of the bioadhesive layer of the bioerodible, water-soluble carrier device.

41. The method of claim 35 or 36 wherein the composition is a solution during the depositing step.

42. The method of claim 35 or 36 wherein the composition is a suspension during the depositing step.

43. The method of claim 35, 36 or 37 wherein the composition is molten during the depositing or affixing step.

44. The method of claim 35 or 37 wherein the composition is a powder during the depositing or affixing step.

45. The method of claim 36 wherein the fluid carrier is a liquid carrier.

46. The method of claim 36 wherein the fluid carrier is a volatile liquid.

47. The method of claim 36 wherein the fluid carrier has a low normal boiling point.

48. The method of claim 36 wherein the fluid carrier is a pharmaceutical solvent suitable for oral administration.

49. The method of claim 36 wherein the fluid carrier comprises acetic acid, acetone, anisole, 1-butanol, 2-butanol, butyl acetate, tertbutylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, or tetrahydrofuran.

50. The method of claim 35, 36 or 37 wherein the composition diffuses into a layer of the bioerodible, water-soluble carrier device.

51. The method of claim 35, 36 or 37 wherein the composition further comprises a viscosity-building agent.

52. The method of claim 35, 36 or 37 wherein the composition comprises more than one active ingredient.

53. The method of claim 35, 36 or 37 wherein the depositing or affixing step is performed more than once.

54. The method of claim 35, 36 or 37 wherein the depositing or affixing step is performed 1 to 10 times.

55. The method of claim 35, 36 or 37 wherein more than one composition is deposited or affixed.

56. The method of claim 35, 36 or 37 wherein 2 to 10 different compositions are deposited or affixed.

57. The method of claim 56 wherein each of the 2 to 10 compositions has a different active ingredient.

58. The method of claim 35, 36 or 37 wherein the composition does not cover the entire surface of a layer.

59. The method of claim 35, 36 or 37 wherein the bioadhesive layer can adhere to a mucosal surface of a mammal.

60. The method of claim 35, 36 or 37 wherein the composition forms a non-bioadhesive deposit after the composition is deposited or affixed to the surface.

61. The method of claim 35, 36 or 37 wherein the composition is deposited near the center of the bioadhesive layer and the periphery of the bioadhesive layer can adhere to a mucosal surface of a mammal.

62. The method of claim 35, 36 or 37 wherein the bioadhesive layer is water-soluble.

63. The method of claim 35, 36 or 37 wherein the bioadhesive layer comprises a film forming water-soluble polymer and a bioadhesive polymer.

64. The method of claim 63 wherein the film forming water soluble polymer of the bioadhesive layer comprises hydroxyethyl cellulose, hydroxypropyl cellulose, hydroxypropylmethyl cellulose, hydroxyethylmethyl cellulose, or a combination thereof.

65. The method of claim 63 wherein the film forming water soluble polymer of the bioadhesive layer is crosslinked or plasticized.

66. The method of claim 63 wherein the bioadhesive polymer of the bioadhesive layer comprises polyacrylic acid, sodium carboxymethyl cellulose or polyvinylpyrrolidone or a combination thereof.

67. The method of claim 66 wherein the polyacrylic acid is partially crosslinked.

68. The method of claim 38 wherein the non-bioadhesive backing layer comprises a pharmaceutically acceptable, water-soluble, film-forming polymer.

69. The method of claim 68 wherein the pharmaceutically acceptable, water-soluble, film-forming polymer is hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, or a combination thereof.

70. The method of claim 68 wherein the pharmaceutically acceptable, water-soluble, film-forming polymer is crosslinked.

71. The method of claim 68 wherein the pharmaceutically acceptable, water-soluble, film-forming polymer comprises hydroxyethyl cellulose and hydroxypropyl cellulose.

72. The method of claim 38 wherein the non-bioadhesive backing layer will dissolve first after application to a mucosal surface of a mammal.

73. The method of claim 35, 36 or 37 wherein the bioerodible, water-soluble carrier device provides sustained delivery of the composition.

74. The method of claim 35, 36 or 37 wherein the composition comprises an andrenergic agent; adrenocortical steroid; adrenocortical suppressant; alcohol deterrent; aldosterone antagonist; amino acid; ammonia detoxicant; anabolic; analeptic; analgesic; androgen; anesthesia; anesthetic; anorectic; antagonist; anterior pituitary suppressant; anthelmintic; anti-acne agent; antiadrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocin; plasminogen activator; platelet activating factor antagonist; platelet aggregaton inhibitor; post-stroke treatment agent; post-head trauma treatment agent; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; agent for treatment of amyotrophic laterial sclerosis; agent for treatment of cerebral ischemia; agent for treatment of Paget's disease; agent for treatment of unstable angina; uricosuric; vasoconstrictor; vasodilator; vulnerary; wound healing agent; or xanthine oxidase inhibitor.

75. The method of claim 35, 36 or 37 wherein the composition comprises Acebutolol; Acebutolol; Acyclovir; Albuterol; Alfentanil; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; or Zolpidem.

76. The method of claim 35, 36 or 37 wherein the composition comprises fentanyl.

77. The method of claim 35, 36 or 37 wherein the composition comprises ondansetron.

78. The method of claim 35, 36 or 37 wherein the composition comprises hydrocodone.

79. The method of claim 35, 36 or 37 wherein the composition comprises between about 0.001 percent and about 30 percent by weight of the bioerodible, water-soluble, carrier device.

80. The method of claim 35, 36 or 37 wherein the composition comprises between about 0.005 percent and about 35 percent by weight of the bioerodible, water-soluble, carrier device.

81. A bioerodible, water-soluble, carrier device made by the method of claim 35, 36 or 37.

82. A method for sustained delivery of a pharmaceutical composition to a mammal that comprises applying a bioerodible, water-soluble, carrier device to a mucosal surface of the mammal, wherein the bioerodible, water-soluble, carrier device comprises a bioadhesive layer and a composition comprising an active ingredient, and wherein the composition is deposited onto a surface of the bioerodible, water-soluble, carrier device after formation of the bioerodible, water-soluble, carrier device.

83. The method of claim 82 wherein the bioerodible, water-soluble, carrier device further comprises a non-bioadhesive backing layer.

84. The method of claim 82 wherein the composition does not cover the entire surface of a layer of the bioerodible, water-soluble, carrier device.

85. The method of claim 82 wherein the bioadhesive layer can adhere to a mucosal surface of a mammal.

86. The method of claim 82 wherein the composition forms a non-bioadhesive deposit.

87. The method of claim 82 wherein the composition is deposited near the center of the surface of the bioadhesive layer and the periphery of the bioadhesive layer can adhere to a mucosal surface of a mammal.

88. The method of claim 82 wherein the composition further comprises a fluid carrier.

89. The method of claim 88 wherein the fluid carrier comprises acetic acid, acetone, anisole, 1-butanol, 2-butanol, butyl acetate, tertbutylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, or tetrahydrofuran.

90. The method of claim 82 wherein the composition further comprises a viscosity-building agent.

91. The method of claim 82 wherein the composition further comprises a polymeric or nonpolymeric hydrophilicity agent.

92. The method of claim 91 wherein the hydrophilicity agent comprises polyethylene glycol.

93. The method of claim 82 wherein the bioadhesive layer is water-soluble.

94. The method of claim 82 wherein the bioadhesive layer comprises a film forming water-soluble polymer and a bioadhesive polymer.

95. The method of claim 94 wherein the film forming water soluble polymer of the bioadhesive layer comprises hydroxyethyl cellulose, hydroxypropyl cellulose, hydroxypropylmethyl cellulose, hydroxyethylmethyl cellulose, or a combination thereof.

96. The method of claim 94 wherein the film forming water soluble polymer of the bioadhesive layer is crosslinked or plasticized.

97. The method of claim 94 wherein the bioadhesive polymer of the bioadhesive layer comprises polyacrylic acid, sodium carboxymethyl cellulose or polyvinylpyrrolidone or a combination thereof.

98. The method of claim 97 wherein the polyacrylic acid is partially crosslinked.

99. The method of claim 83 wherein the non-bioadhesive backing layer comprises a pharmaceutically acceptable, water-soluble, film-forming polymer.

100. The method of claim 99 wherein the pharmaceutically acceptable, water-soluble, film-forming polymer is hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, or a combination thereof.

101. The method of claim 99 wherein the pharmaceutically acceptable, water-soluble, film-forming polymer is crosslinked.

102. The method of claim 99 wherein the pharmaceutically acceptable, water-soluble, film-forming polymer comprises hydroxyethyl cellulose and hydroxypropyl cellulose.

103. The method of claim 82 wherein the composition is a liquid composition when deposited onto the surface.

104. The method of claim 82 wherein the composition is a solid composition when deposited onto the surface.

105. The method of claim 82 wherein the composition is a molten composition when deposited onto either layer.

106. The method of claim 82 wherein the composition is deposited onto either layer more than once.

107. The method of claim 82 wherein the composition is deposited onto either layer between about 1 to about 10 times.

108. The method of claim 83 wherein the non-bioadhesive backing layer will dissolve first.

109. The method of claim 82 wherein the composition comprises an andrenergic agent; adrenocortical steroid; adrenocortical suppressant; alcohol deterrent; aldosterone antagonist; amino acid; ammonia detoxicant; anabolic; analeptic; analgesic; androgen; anesthesia; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocin; plasminogen activator; platelet activating factor antagonist; platelet aggregaton inhibitor; post-stroke treatment agent; post-head trauma treatment agent; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; agent for treatment of amyotrophic laterial sclerosis; agent for treatment of cerebral ischemia; agent for treatment of Paget's disease; agent for treatment of unstable angina; uricosuric; vasoconstrictor; vasodilator; vulnerary; wound healing agent; or xanthine oxidase inhibitor.

110. The method of claim 82 wherein the composition comprises Acebutolol; Acebutolol; Acyclovir; Albuterol; Alfentanil; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; or Zolpidem.

111. The method of claim 82 wherein the composition comprises fentanyl.

112. The method of claim 82 wherein the composition comprises ondansetron.

113. The method of claim 82 wherein the composition comprises hydrocodone.

114. The method of claim 82 wherein the composition comprises between about 0.001 percent and about 50 percent by weight of the bioerodible, water-soluble, carrier device.

115. The method of claim 82 wherein the composition comprises between about 0.005 percent and about 35 percent by weight of the bioerodible, water-soluble, carrier device.
